(19) United States
(12) Patent Application Publication
(10) Pub. No.: US 2025/0104403 A1
(43) Pub. Date: Mar. 27, 2025

(54) IMPROVED DELINEATION OF IMAGE LEVEL ANNOTATION, FOR INSTANCE FOR ACCURATE TRAINING OF MEDICAL IMAGE SEGMENTATION MODELS

(71) Applicant: Siemens Healthineers AG, Forchheim (DE)

(72) Inventors: Punith B V, Hassan (IN); Asha K.K, Bangalore (IN)

(73) Assignee: Siemens Healthineers AG, Forchheim (DE)

(21) Appl. No.: 18/894,210

(22) Filed: Sep. 24, 2024

(30) Foreign Application Priority Data
Sep. 27, 2023 (DE) 10 2023 209 430.4

(51) Int. Cl. G06V 10/774; A61B 6/00; G06V 10/26; G06V 10/50; G06V 10/74; G06V 20/70

(52) U.S. Cl. CPC G06V 10/774; G06V 10/26; G06V 10/507; G06V 10/761; G06V 20/70; A61B 6/5294; G06V 2201/03

(57) ABSTRACT

A method for refining annotations in medical images, comprises: obtaining an initially annotated image; cropping said initially annotated image to obtain a cropped image, which retains only a part of the initially annotated image indicated by the annotation; analyzing pixel intensity distributions within the cropped image; segmenting the cropped image based on the analysis of the pixel intensity distributions to obtain a segmented image; refining the segmented image to obtain a refined segmented image; and performing similarity matching on the refined segmented image to obtain a delineation mask.

### CROSS-REFERENCE TO RELATED APPLICATION(S)

The present application claims priority under 35 U.S.C. § 119 to German Patent Application No. 10 2023 209 430.4, filed Sep. 27, 2023, the entire contents of which is incorporated herein by reference.

### FIELD

One or more embodiments of the present invention relate to the field of medical imaging and, more particularly, to a method for refining image annotations for preparing these images as training data for a machine learning model. An aim is to improve the training of medical image segmentation models using deep learning. The solution proposed can be also used as a standalone tool to refine the standard designation of a region of interest (ROI) on an image annotated, such that the annotation better fits a targeted zone such as a pathology. This also helps for proper analysis of the annotated medical images.

### BACKGROUND

In recent years, the technologies of artificial intelligence have been experiencing significant advancements. One of the most promising fields of use is the application of this technology for medical image processing. This transformation has led to the automation of many tasks that were previously manual that is to say performed by a skilled human (from reading, to annotating or even diagnosing), resulting in more efficient processes. However, for the success of machine learning and deep learning methods in this field, there is a need for accurate training data, that is to say that the availability of medical images with accurate annotations for being reference and test data for training the model is of utmost importance.

Traditionally, the gold standard method for producing these reference annotations has been manual annotation, that is to say an annotation that is performed by a skilled human being (technician or medical practitioner). While it might be considered the most accurate, manual annotation has its set of challenges. First, it necessitates the involvement of trained professionals, such as technicians, radiologists or pathologists, who bring their expertise to the task. However, this manual process is inherently time-consuming and can introduce variability, as interpretations can differ among professionals. For specific medical issues such as the annotation of White Matter Hyperintensities (WMH), the lack of comprehensive guidelines exacerbates this variability. This challenge is further amplified in a federated learning context, where annotations from multiple annotators at different sites are very likely to introduce a higher interpretation variability.

Moreover, for artificial intelligence models, especially deep learning-based ones, periodic updates are crucial to accommodate new variations and incorporate feedback from users. A common practice involves gathering feedback annotations from radiologists or pathologists during their routine use of the application. However, given their primary responsibilities and constraints on time, these professionals often provide rough annotations, such as bounding boxes or freehand annotations, instead of detailed ones. Unfortunately, these rough annotations on image are not precise enough for direct use for retraining the models. In most cases, a team of skilled technician must rework the annotated image data provided as feedback by the medical practitioners in order to obtain a more accurate annotated image which can be used as training data for the model. Such an approach requires training skilled technicians and is very time consuming. In addition, it is error and variation prone.

### SUMMARY

The inventors have identified a need for an automated approach for bridging this gap, efficiently converting rough or image-level annotations from medical practitioners into detailed voxel-level annotations that can be used for the training of the model. One or more embodiments of the present invention aim at addressing at least this need, introducing an innovative automated approach for refinement of medical data with annotation.

One or more embodiments of the present invention present an innovative approach for refining and enhancing medical image annotations, specifically transitioning from the rough or image-level annotations commonly provided by medical professionals to more precise voxel-level annotations that are crucial for training robust deep learning models. It is to be noted that the solution proposed for changing (improving the precision of the annotation) can also be used as a standalone tool to refine the standard designation of a region of interest (ROI) on an image annotated, such that the annotation better fits a targeted zone such as a pathology. This also helps for proper analysis of the annotated medical images.

The process commences by focusing on the areas or regions of interest through image cropping, taking the freehand annotations or bounding boxes around pathologies provided by radiologists or pathologists as a reference. This initial cropping step serves a dual purpose: it emphasizes the target pathology and significantly reduces the initial computational load by limiting the volume of data to be processed in subsequent steps.

Following this, a histogram analysis of the cropped area is executed. This analysis allows for discerning between different image objects based on their pixel intensity distributions. By analyzing these histogram modes and integrating them with existing knowledge about the image—such as the specific organ in question, the image modality, or the imaging sequence—interval threshold values are determined. These values play an important role in segmenting the image, distinguishing the pathology or region of interest (ROI) from other surrounding structures.

The segmented image, which results from the application of these thresholds, then becomes input data for an Expectation Maximization algorithm, a tool employed to generate an initial refined segmentation. For greater flexibility, there is also an option to use a Convolutional Neural Network (CNN) model for this step.

Another novel aspect of embodiments of the present invention is its incorporation of a Structural Similarity Index (SSIM) calculation. Using tools like the ‘scikit-image’ package from Python, the SSIM between the newly generated cropped volume or segmentation and similar structures in a training set is computed. The SSIM metric, which ranges between −1 to 1, offers a quantitative measure of similarity, with a value of 1 indicating a perfect match.

Leveraging this similarity metric, the segmented structure from the training set with the highest SSIM is used to fine-tune and refine the segmentation produced in the previous steps. This could involve processes such as edge smoothing to ensure the final annotation is as accurate as possible.

In practical applications, using embodiments of the present invention has led to successfully convert rough annotations of structures like kidneys from CT images or white matter hyperintensities from FLAIR and MPRAGE images into highly accurate voxel-level annotations. The implications of one or more embodiments of the present invention are profound: it offers a cost-effective, time-efficient solution for generating high-quality annotations, drastically reduces the variability introduced by different annotators, reduces errors and reduces the computer resources needed (storage and computing power needed).

### BRIEF DESCRIPTION OF THE DRAWINGS

The details of one or more embodiments are set forth in the accompanying drawings and the description below. Other features will be apparent from the description and drawings, and from the claims.

FIG. 1 is a schema illustrating the traditional approach for performing the updated training of the model.

FIG. 2 is a schema illustrating the general idea of the approach in an embodiment of the present invention.

FIG. 3 is a diagram of a method for obtaining training data from rough annotated data.

FIG. 4 illustrates several steps (a) to (f) of the method for automatic annotation aiming at obtaining a voxel level annotation according to an embodiment of the present invention starting from a rough annotation of a kidney from CT image.

FIG. 5 illustrates several steps (a) to (f) of the method for automatic annotation aiming at obtaining a voxel level annotation according to an embodiment of the present invention starting from a rough annotation of WMH from FLAIR image.

FIG. 6 illustrates several steps (a) to (f) of the method for automatic annotation aiming at obtaining a voxel level annotation according to an embodiment of the present invention starting from a rough annotation of WMH from MPRAGE image.

FIG. 7 illustrates the start (a) and the result (b) of the method for automatic annotation aiming at obtaining a voxel level annotation according to an embodiment of the present invention starting from a rough ellipse annotation of WMH.

### DETAILED DESCRIPTION

The terms used in this specification were selected to include current, widely used general terms. In certain cases, a term may be one that was arbitrarily established by the applicant. In such cases, the meaning of the term will be defined in the relevant portion of the detailed description. As such, the terms used in the specification are not to be defined simply by the name of the terms but are to be defined based on the meanings of the terms as well as the overall description of the present disclosure.

Hereinafter, embodiments of the present invention will be described in detail with reference to the accompanying drawings. It should be noted that the same reference numerals are used to designate the same or similar elements throughout the drawings.

It should also be noted that the present application relates to medical images, however, its teaching could be used in any type of image comprising a rough image level annotation that needs to be refined to obtain a pixel or voxel level annotation. The description refers mainly to three types of medical imaging technologies: CT, FLAIR and MPRAGE. However, the teaching of the present invention is not limited to using these types of medical images.

Computed Tomography, often known as CT or CAT scan, is a medical imaging technique that produces detailed pictures of the body's internal structures by taking a series of X-ray images from different angles. This method allows for the creation of cross-sectional images that can be used to examine various internal organs, bones, soft tissues, and blood vessels with greater detail than traditional X-rays.

Fluid-Attenuated Inversion Recovery, commonly referred to as FLAIR, is a particular sequence used in magnetic resonance imaging (MRI). The main purpose of FLAIR is to suppress the signal from fluids, especially the cerebrospinal fluid found in the brain. By doing so, it makes abnormalities like multiple sclerosis plaques or some strokes, which are surrounded by this fluid, more visible and easier to identify.

Magnetization Prepared Rapid Acquisition Gradient Echo, abbreviated as MPRAGE, is another sequence used in MRI, specifically designed to produce detailed, T1-weighted images of the brain. This imaging technique is especially beneficial for capturing fine details of brain structures. Often, MPRAGE images are utilized in studies that require detailed structural brain imaging, such as research into neurodegenerative diseases.

One or more embodiments of the present invention revolves around a comprehensive method to refine image annotations in the domain of medical imaging. The primary objective is to transition from image-level annotations, typically coarse and general, to accurate voxel-level annotations, which are more precise and detailed, using an amalgamation of classic image processing techniques and available training data.

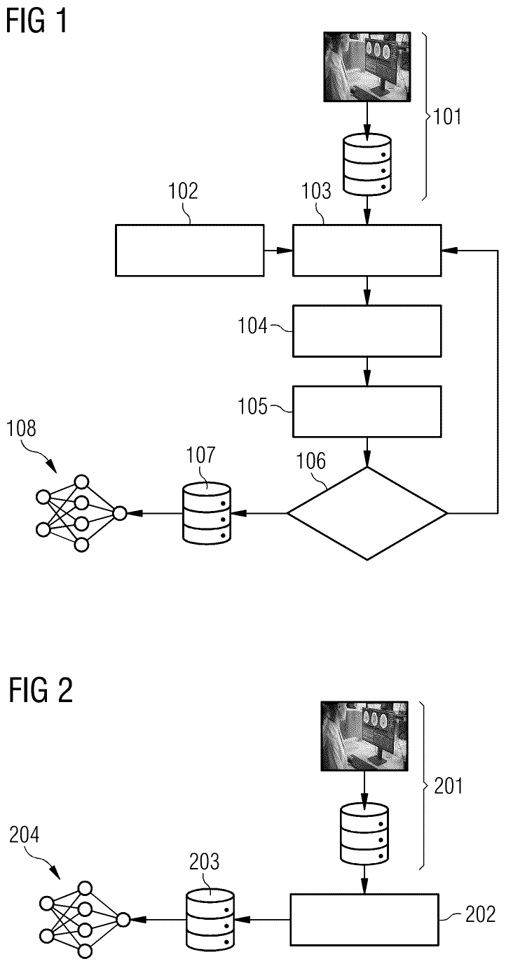

FIG. 1 illustrates the conventional approach according to traditional art.

At step 101 a radiologist or medical practitioner performs rough annotation on a medical image. This annotation can be done for instance on a computer using a mouse or on a tablet using a pencil or using any other type of rough selection performed from the user interface of a computer of any type. The image data can be a two-dimensional or three-dimensional image. In the case of a two-dimensional, the medical practitioner can make a rough selection by drawing an ellipse, a circle, a square or any two-dimensional geometrical figure which is defining a perimeter for delineating the region of interest (ROI) in the image. This ROI can be for the illustration of a pathology or the identification of any specific element in the image. The image data can also be a three-dimensional image. In that case, the selection can be performed the same way with the computer extruding 3D volume from a 2D input or by any other kind of selection of a volume in the 3D image via known tools for human machine interactions. The obtained annotated image data is then stored.

At step 102, annotators can be trained to be capable of refining the annotations in the image data. Alternatively, or in addition computer technicians can be trained to be capable of performing this refinement of the annotation.

At step 103, the train annotators or technicians will perform the modification of the annotation.

At step 104, annotation corrections are performed by radiographers.

At step 105, the annotations are reviewed by clinical experts.

At step 106, the annotation consensus is check using predefined thresholds. In case with the annotation consensus is below the thresholds, step 103 is restarted. In case this check is successful, the refined training data is stored at step 107. At step 108, this data can be used for retraining the model.

This approach is essentially based on human expertise from the medical practitioners making rough annotations, from the trained annotators that are performing modifications and corrections and from the clinical experts performing the review of the annotations. In other words, this approach is very time consuming, error prone and subject to a lot of variations because of the number and variety of people involved. Further, it requires at every step of the process storing important amounts of data corresponding to the work in progress data, performing several steps of computations and performing several transfers of these working progress data. That is to say that also on a purely computer technical point of view, this approach entails several drawbacks linked to an inefficient use of the computer resources.

FIG. 2 illustrates the approach according to present invention. The step 201 is similar to the step 101 performed in the method of conventional art illustrated in FIG. 1.

However, at step 202, the annotation refinement tool implemented on a computer is used on the roughly annotated image data as input for obtaining—after computing—accurate voxel level annotated image as output.

The steps 203 of storage and 204 of retraining the model are similar to the step 107 and 108 discussed above for the conventional approach.

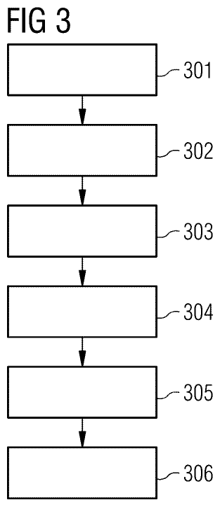

FIG. 3 shows a diagram of a method for obtaining training data from roughly annotated data according to an embodiment of the present invention.

In a first step 301, an Image Cropping step, the roughly annotated image data is worked such that the image level annotations provided by the radiologists or medical practitioners (for instance as free hand annotation, see step 101 and 201 discussed above) leads to a cropping. This step streamlines the subsequent processes as the regions of interest (two-dimensional surfaces or three-dimensional volumes) annotated by the radiologists or pathologists are cropped to concentrate on the pathology or elements of interest in the image. By focusing on this cropped region, areas of non-interest are discarded, leading to a more efficient and accurate annotation refinement process, but also reducing the amount of data stored and processed in subsequent steps of the method.

A second step 302, following the cropping is the Multimodal Histogram Analysis of the cropped region.

Multimodal histogram analysis is a method used to understand the distribution of pixel intensities in an image (or voxel in case of a 3D image). When the distribution of these pixel intensities is graphically represented, the resulting graphical depiction is termed a histogram. Distinctively, a histogram that showcases multiple prominent peaks, or “modes”, suggests the presence of several primary groups of pixel intensities in the image.

In the field of medical imaging, these modes can be emblematic of diverse tissue types or specific anatomical structures or pathologies). For instance, a certain peak might correlate to the intensity values characteristic of bony tissue, while another distinct peak might be indicative of adipose tissue or a tumor. Such analysis aids in determining the range of pixel intensities associated with specific anatomical or pathological structures, facilitating the differentiation or “segmentation” of these structures within the image. Furthermore, when images derived from disparate imaging modalities, such as Magnetic Resonance Imaging (MRI) and Computed Tomography (CT), are concurrently assessed, each modality contributes its unique set of pixel intensity values. A comprehensive analysis of aggregated histograms can yield augmented insights regarding the anatomical and pathological structures under scrutiny.

In embodiments of the present invention, this Multimodal Histogram Analysis delves into the distribution of pixel or voxel values within the cropped surface or volume. By examining the modes in the histogram, distinct image objects, such as organs, tumors, and background regions, can be identified.

A third step 303 is an Interval Threshold Determination step.

This step is intricately tied to the multimodal histogram analysis step performed on the region of interest within the image that has been cropped. Its primary purpose is to achieve distinct and accurate demarcation of the structures or entities present in that specific region. Upon isolating the desired region by cropping the image around the annotations provided by a radiologist or pathologist, the histogram of this cropped region is thoroughly examined. This examination seeks to identify the distinct modes within the histogram that correspond to different structures, such as organs or abnormalities like tumors, against the background areas. For performing this step, the process integrates available prior knowledge about the image in question. This could encompass specific attributes of the organ or pathology being viewed, the type of imaging modality used (like CT or MRI), and potentially the unique sequence of the image if that is relevant. A data base with image information on organ, modality and sequence can be used to feed data in this step.

The method then either obtains an input from a user to set appropriate threshold values around the detected modes or automatically determines this appropriate threshold values. These values effectively act as demarcation lines within the histogram. They differentiate between pixel intensities that correspond to distinct structural entities within the image. As an illustrative example, if an image showcases a tumor with higher intensity values than the surrounding tissue, the selected thresholds would optimally lie between these two intensity groupings, enabling precise segmentation of the tumor.

This segmented image, created using determined threshold values, subsequently serves as a starting point for a fourth step 304 using an Expectation Maximization (EM). It is employed to enhance the precision of the preliminary image segmentation derived from the threshold values. This refinement improves the result of the preliminary segmentation is particularly adapted due to the inherent challenges posed by medical images, such as variability in tissue contrasts and potential artifacts. Initially, the segmented image from the thresholding process serves as a foundational guess or prior for the EM algorithm. This preliminary segmentation provides a starting point for the EM's optimization process. The EM algorithm operates through two primary iterative steps: the Expectation step and the Maximization step.

In the Expectation step, for each pixel in the image, the algorithm computes the probability that the pixel belongs to a particular segment or structure. This is based on model parameters, effectively assigning soft or probabilistic labels to pixels, hinting at their likely classifications.

The Maximization step follows, leveraging the probabilities determined in the Expectation step to adjust and refine the model parameters. The aim of this phase is to tweak these parameters to provide the most fitting explanation for the observed data, pushing the segmentation towards higher accuracy.

This loop of estimating probabilities and then maximizing the fit based on those probabilities is executed repeatedly. As it cycles through, the segmentation of the image becomes increasingly refined. This iterative process concludes when there is minimal change in model parameters between iterations, signaling that an optimal segmentation solution has been achieved.

In cases where more advanced methods are needed, a Convolutional Neural Network (CNN) model can be trained to generate the initial segmentation, taking advantage of available prior data.

To ensure the quality and accuracy of the generated segmentation, the method comprises a fifth step 305 of performing a similarity matching. This is achieved through using “Structural Similarity Index (SSIM) Computation”. This is performed by using the initial training data of the model or by leveraging know data for instance the “scikit-image” package from Python, the SSIM between the newly generated cropped volume or initial segmentation and similar structures found in the training set is then computed. The SSIM offers a range from −1 to 1, with a value of 1 signifying a perfect match.

Lastly, with the aim of maximizing the quality of the annotations, the ‘Fine-tuning’ step 307 is performed. Here, the structure from the training dataset that has the highest SSIM value, indicating the closest match, is used. The segmentation mask generated in the previous steps is refined and optimized using this structure, ensuring annotations of the highest precision.

In real-world applications, this method has shown impressive outcomes. For instance, rough annotations of kidneys from CT images were seamlessly converted to voxel-level annotations. Similarly, white matter hyperintensities (WMH) annotations from both FLAIR and MPRAGE images were meticulously refined to voxel-level using the described steps.

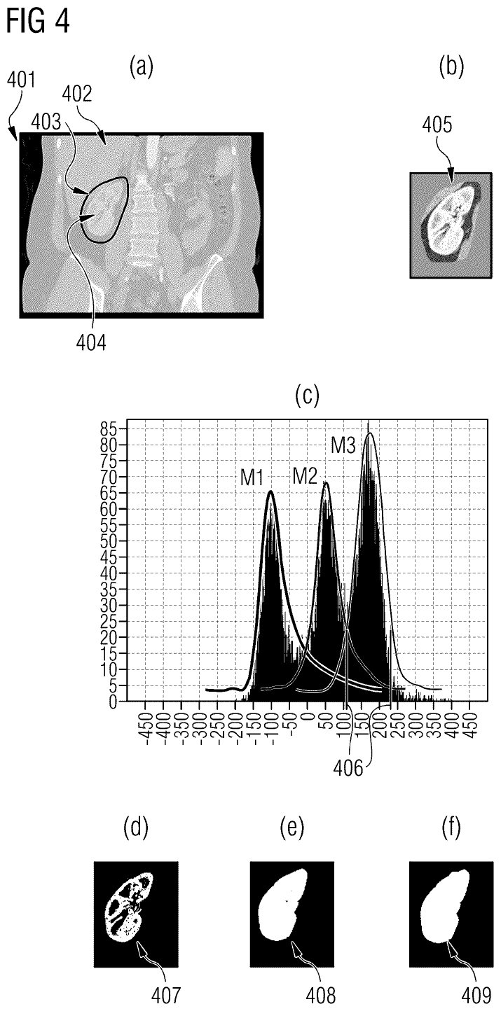

FIG. 4 is an illustration of such a real-world application of the method for automatic annotation starting from a rough annotation of a kidney from CT image.

The sub-FIG. 4(a) shows a CT image 401 of a human body 402 focused on the pelvic region. A rough annotation 403 has been performed around a kidney 404. This roughly annotated image is used an input for the method illustrated a FIG. 3.

The sub-FIG. 4(b) shows a cropped image 405 (so an image of the cropped data) of the region of interest defined by the rough annotation.

The sub-FIG. 4(c) shows the result of the histogram analysis with distributions of pixel intensities. Three modes M1, M2 and M3 are identifiable as being three peaks in the histogram. For this image, the object of interest is bright, this entails that the mode with the brightest intensity is used. Threshold values 406 can be set on this mode for instance by user selection.

The sub-FIG. 4(d) shows the pixels selected for the segmented image 407.

The sub-FIG. 4(e) shows the mask 408 obtained by the method of an embodiment of the present invention whilst the sub-FIG. 4(f) shows the mask according to the ground true 409.

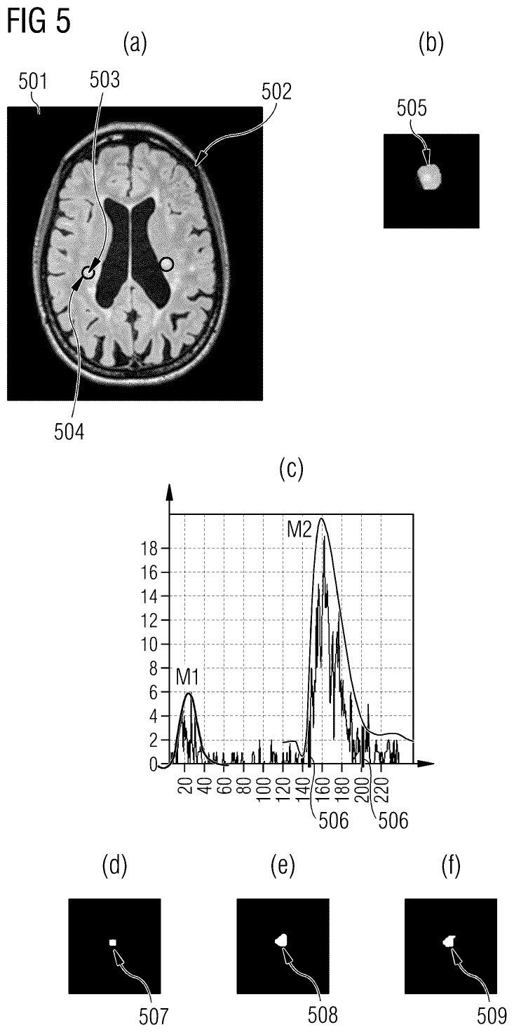

FIG. 5 is another illustration of such a real-world application of the method for automatic annotation starting from a rough annotation of WMH from FLAIR image.

The sub-FIG. 5(a) shows a FLAIR image 501 of a human body 502 focused on the skull and brain region. A rough annotation 504 has been performed around a white matter hyperintensities (WMH) 503. This image is used an input for the method illustrated a FIG. 3.

The sub-FIG. 5(b) shows a cropped image 505 (so an image of the cropped data) of the region of interest defined by the rough annotation.

The sub-FIG. 5(c) shows the result of the histogram analysis with distributions of pixel intensities. Two modes M1 and M2 are identifiable as being two peaks in the histogram. For this image the object of interest is bright, this entails that the mode with the brightest intensity is used. Threshold values 506 can be set on this mode for instance by user selection.

The sub-FIG. 5(d) shows the pixels selected for the segmented image 507.

The sub-FIG. 5(e) shows the mask 508 obtained by the method of an embodiment of the present invention whilst the sub-FIG. 5(f) shows the mask according to the ground true 509.

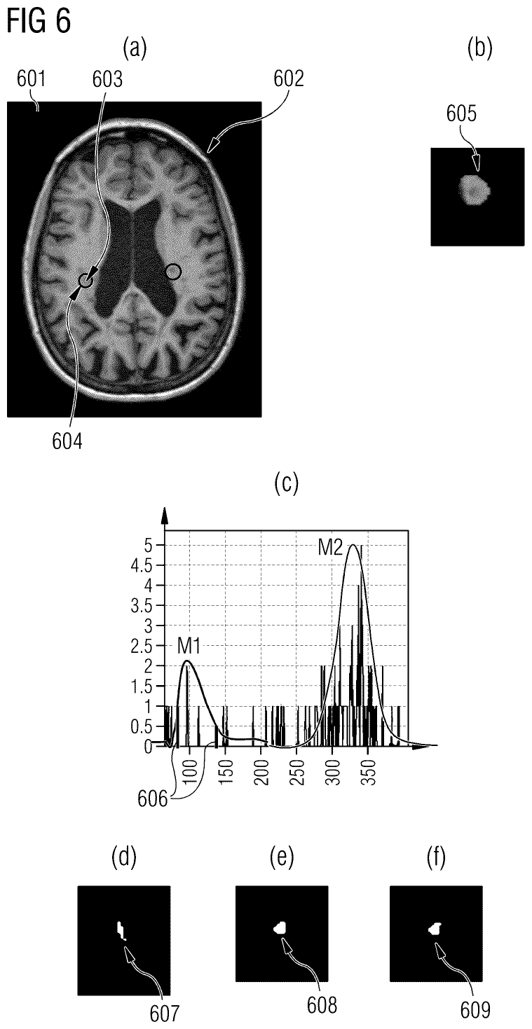

FIG. 6 is another illustration of such a real-world application of the method for automatic annotation starting from a rough annotation of WMH from MPRAGE image.

The sub-FIG. 6(a) shows a MPRAGE image 601 of a human body 602 focused on the skull and brain region. A rough annotation 604 has been performed around a white matter hyperintensities (WMH) 603. This image is used an input for the method illustrated a FIG. 3.

The sub-FIG. 6(b) shows a cropped image 605 (so an image of the cropped data) of the region of interest defined by the rough annotation.

The sub-FIG. 6(c) shows the result of the histogram analysis with distributions of pixel intensities. Two modes M1 and M2 are identifiable as being two peaks in the histogram. For this image, the object of interest is dark, this entails that the mode with the least bright intensity is used. Threshold values 606 can be set on this mode for instance by user selection.

The sub-FIG. 6(d) shows the pixels selected for the segmented image 607.

The sub-FIG. 6(e) shows the mask 608 obtained by the method of an embodiment of the present invention whilst the sub-FIG. 6(f) shows the mask according to the ground true 609.

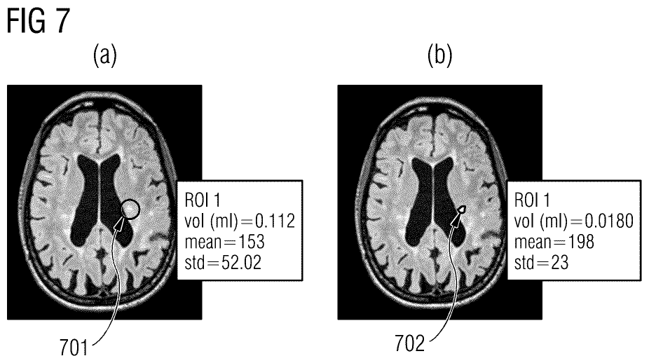

FIG. 7 illustrates the start and result for a roughly annotated FLAIR image. The start is shown by the FIG. 7(a) with an ellipse 701 and the result is shown in the FIG. 7(b) with the obtaining of a voxel level annotation 702 of the WMH. In this case, it is to be noted that this also shows how the possibility to use the solution as a standalone tool as mentioned above.

The refined image data with voxel level annotation thus obtained has the necessary feature for being used in the retraining of the model.

In embodiments of the present invention, the model is re-trained using the newly obtained refined image data and the model is used for diagnostic.

In conclusion, one or more embodiments of the present invention address the need for precise and consistent annotations. Traditional methods of manual annotations, while regarded as the gold standard, are time-consuming and can introduce significant interpretation variability. This is particularly pronounced in the domain of federated learning which is mostly used for medical image annotation.

Embodiments of the present invention offer an innovative approach for refining rough, image-level annotations to generate precise voxel-level annotations. Through the innovative combination of traditional image processing techniques and modern deep learning methodologies, embodiments of the present invention successfully transform general image-level indications into detailed voxel-level annotations. This breakthrough not only assures enhanced accuracy but also significantly diminishes the time and cost associated with the annotation process.

Furthermore, the adoption of one or more embodiments of the present invention ensures a dramatic reduction in interpretation variability, ultimately leading to consistent and reliable model performances. The quality of annotations, a cornerstone for the efficacy of Deep Learning methodologies, is significantly elevated, translating into superior model performance outcomes.

Notably, the methods outlined in accordance with one or more embodiments of the present invention also yields a substantial enhancement in the efficiency of computer resource utilization. The reduced computational overhead resulting from one or more embodiments of the present invention ensures that computer resources, often a limitation in large-scale medical imaging analyses, are used more effectively and efficiently. Indeed, one or more embodiments of the present invention limit the storage of in progress work image and limit the storage and computation to segmented images.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A method for refining annotations in images, the method comprising:
obtaining an initially annotated image;
cropping said initially annotated image to obtain a cropped image, which retains only a part of the initially annotated image indicated by the annotation;
analyzing pixel intensity distributions within the cropped image;
segmenting the cropped image based on the analysis of the pixel intensity distributions to obtain a segmented image;
refining the segmented image to obtain a refined segmented image; and
performing similarity matching on the refined segmented image to obtain a delineation mask.

2. The method of claim 1, wherein the annotation in the initially annotated image is an image-level annotation.

3. The method of claim 1, wherein the initially annotated image is a medical image.

4. The method of claim 1, wherein the cropping comprises:
retaining only the part of the initially annotated image to focus on a region of interest defined by the annotation, the part of the initially annotated image being inside a perimeter defined by the annotation, and
removing a part of the initially annotated image which is outside the perimeter defined by the annotation.

5. The method of claim 1, wherein the analyzing comprises:
performing a histogram analysis on pixel intensities within the cropped image.

6. The method of claim 5, wherein the performing a histogram analysis on pixel intensities within the cropped image comprises:
detecting modes and determining threshold values.

7. The method of claim 6, wherein the segmenting comprises:
segmenting the cropped image based on the threshold values.

8. The method of claim 1, wherein the refining the segmented image comprises:
using an iterative Expectation Maximization algorithm.

9. The method of claim 8, wherein the iterative Expectation Maximization algorithm uses the segmented image as an initial or prior guess.

10. The method of claim 9, wherein the using of the iterative Expectation Maximization algorithm comprises:
calculating a probability that each pixel belongs to a particular segment or structure, and
adjusting model parameters based on probabilities derived from the calculating.

11. The method of claim 1, wherein the performing of the similarity matching on the refined segmented image comprises:
using a Structural Similarity Index computation and performing a fine-tuning where a structure from a training dataset with a highest Structural Similarity Index value is utilized to optimize the delineation mask.

12. The method of claim 1, wherein the delineation mask is a voxel-level annotation.

13. A data processing system comprising:
a processor configured to perform the method of claim 1.

14. A non-transitory computer program product comprising instructions, wherein when the instructions are executed by a computer, the instructions cause the computer to carry out the method of claim 1.

15. A non-transitory computer-readable storage medium storing instructions that, when executed by a computer, cause the computer to carry out the method of claim 1.

16. The method of claim 2, wherein the cropping comprises:
retaining only the part of the initially annotated image to focus on a region of interest defined by the annotation, the part of the initially annotated image being inside a perimeter defined by the annotation, and
removing a part of the initially annotated image which is outside the perimeter defined by the annotation.

17. The method of claim 16, wherein the analyzing comprises:
performing a histogram analysis on pixel intensities within the cropped image.

18. The method of claim 4, wherein the analyzing comprises:
performing a histogram analysis on pixel intensities within the cropped image.

19. The method of claim 4, wherein the refining the segmented image comprises:
using an iterative Expectation Maximization algorithm.

20. The method of claim 4, wherein the performing of the similarity matching on the refined segmented image comprises:
using a Structural Similarity Index computation and performing a fine-tuning where a structure from a training dataset with a highest Structural Similarity Index value is utilized to optimize the delineation mask.
